(19) United States
(12) Patent Application Publication
(10) Pub. No.: US 2001/0044699 A1
(43) Pub. Date: Nov. 22, 2001

(54) Predicting metabolic stability of drug molecules

(76) Inventors: Todd J. A. Ewing, Newark, CA (US); Paresh I. Patel, East Palo Alto, CA (US); Hung Tieu, Alameda, CA (US); Kenneth R. Korzekwa, Mountain View, CA (US)

Correspondence Address: BEYER WEAVER & THOMAS LLP, P.O. BOX 778, BERKELEY, CA, 94704-0778, US

(21) Appl. No.: 09/811,283

(22) Filed: Mar. 15, 2001

Related U.S. Application Data
(63) Continuation-in-part of application No. 09/613,875, filed on Jul. 10, 2000.
Application No. 09/613,875 is a continuation-in-part of application No. 09/368,511, filed on Aug. 5, 1999.
(60) Provisional application No. 60/217,227, filed on Jul. 10, 2000.

(51) Int. Cl. G06F 19/00; G01N 33/48; G01N 33/50; G01N 31/00

(52) U.S. Cl. 702/19; 702/27

(57) ABSTRACT

Methods are disclosed for developing models used to rapidly predict metabolic stability and regioselectivity of drug molecules. Training sets, based on a sample of molecules with known reaction rates and/or activation energies, are used along with structural descriptors of the molecules in order to develop mathematical models of metabolism based on regression analysis of the activation energies and descriptors. The resulting models are then used to predict the metabolism of other molecules. The invention is particularly useful in developing simple models of cytochrome p450 enzyme metabolism.

### CROSS-REFERENCE TO RELATED APPLICATIONS

This patent application is a continuation-in-part of U.S. patent application Ser. No. 09/368,511, “Use of Computational and Experimental Data to Model Organic Compound Reactivity in Cytochrome p450 Mediated Reactions and to Optimize the Design of Pharmaceuticals,” filed Aug. 5, 1999 by Korzekwa et al. (Atty Docket No.: CAMIP001), U.S. patent application Ser. No. 09/613,875, “Relative Rates of Cytochrome p450 Metabolism,” filed Jul. 10, 2000 by Korzekwa et al. (Atty Docket No.: CAMIP002), and U.S. Provisional Patent application No. 60/217,227, “Accessibility Correction Factors for Quantum Mechanical and Molecular Models of Cytochrome p450 Metabolism,” filed Jul. 10, 2000 by Ewing et al. (Atty Docket No.: CAMIP004P). These patent applications, as well as any other patents, patent applications and publications cited herein, are hereby incorporated by reference in their entirety for all purposes.

### FIELD OF THE INVENTION

The present invention relates generally to systems and methods for developing quantum chemical learning systems used to predict metabolic stability and regioselectivity of drug molecules. Training sets, based on a sample of molecules with known reaction rates and activation energies, are used along with descriptors of the molecules in order to develop mathematical models of metabolism based on regression analysis of the training sets and descriptors. The learning systems are then used to predict the metabolism of other molecules. The invention is particularly useful in developing a model of cytochrome p450 enzyme metabolism.

### BACKGROUND OF THE INVENTION

Drug development is an extremely expensive and lengthy process. The cost of bringing a single drug to market is about $500 million to $1 billion dollars, with the development time being about 8 to 15 years. Drug development typically involves the identification of 1000 to 100,000 candidate compounds distributed across several compound classes that eventually lead, to a single or several marketable drugs.

Those thousands of candidate compounds are screened against biochemical targets to assess whether they have the pharmacological properties that the researchers are seeking. This screening process leads to a much smaller number of “hits” (perhaps 500 or 1000) which display some amount of the desired properties, which are narrowed to even fewer “leads” (perhaps 50 or 100) which are more efficacious. At this point, typically, the lead compounds are assayed for their ADME/PK (absorption, distribution, metabolism, elimination/pharmokinetic) properties. They are tested using biochemical assays such as Human Serum Albumin binding, chemical assays such as pK_{A} and solubility testing, and in vitro biological assays such as metabolism by endoplasmic reticulum fractions of human liver, in order to estimate their actual in vivo ADME/PK properties. Most of these compounds are discarded because of unacceptable ADME/PK properties.

In addition, even optimized leads that have passed these tests and are submitted for FDA clinical trials as investigational new drugs (INDs) will sometimes show undesirable ADME/PK properties when actually tested in animals and humans. Abandonment or redesign of optimized leads at this stage is extremely costly, since FDA trials require formulation, manufacturing and extensive testing of the compounds.

The development of compounds with unacceptable ADME/PK properties thus contributes greatly to the overall cost of drug development. If there was a process by which compounds could be discarded or redesigned at an earlier stage of development (the earlier the better), then great savings in terms of money and time could be achieved. The current art essentially offers no comprehensive method by which this can be done.

A large portion of all drug metabolism in humans and most all organisms is carried out by the cytochrome p450 enzymes. The cytochrome p450 enzymes (CYP) are a superfamily of heme-containing enzymes that include more than 700 individual isozymes that exist in plant, bacterial and animal species. Nelson et al. Pharmacogenetics 1996 6, 1-42. They are monooxygenase enzymes. Wislocki et al., in Enzymatic Basis of Detoxification (Jakoby, Ed.), 135-83, Academic Press, New York, 1980. Although humans share the same several CYP isozymes, these isozymes can vary slightly between individuals (alleles) and the isozyme profile of individuals, in terms of the amount of each isozyme that is present, also varies to some degree.

It is estimated that in humans, 50% of all drugs are metabolized partly by the p450 enzymes, and 30% of drugs are metabolized primarily by these enzymes. The most important CYP enzymes in drug metabolism are the CYP3A4, CYP2D6 and CYP2C9 isozymes. While modeling techniques do exist for predicting substrate metabolism by enzymes other than CYP, no sufficiently accurate technique exists for modeling metabolism by the CYP enzymes. To the extent that modeling techniques are available for other enzymes, they work by analyzing the either the interactions between enzyme and substrate, or the common characteristics for a series of substrates. See, for example, Schramm, “Enzymatic transition states and transition state analog design.” Annu Rev Biochem 1998; 67: 693-720; Hunter, “A structure-based approach to drug discovery; crystallography and implications for the development of antiparasite drugs.” Parasitology 1997; 114 Suppl: S17-29; Gschwend et al, “Molecular docking towards drug discovery.” Mol Recognit 1996 March-April; 9(2): 175-86.

While these modeling techniques are partially effective for some enzymes, they can be ineffective for the CYP enzymes. This is because the CYP enzymes do not have binding specificities in the way that other enzymes do. CYP3A4 is almost completely nonspecific from a steric perspective, while CYP2D6 and CYP2C9 are only modestly sterically specific. Gross steric and electrostatic properties of a substrate have a secondary effect on their metabolism by the CYP enzymes, at most. Thus modeling techniques in the current art cannot be used to model CYP enzyme metabolism.

Systems and methods that provide effective models of substrate metabolism are disclosed in U.S. patent application Ser. No. 09/368,511 (Atty Docket No.: CAMIP001), U.S. patent application Ser. No. 09/613,875 (Atty Docket No.: CAMIP002). Systems and methods that provide accessibility correction factors for the quantum mechanical models are disclosed in U.S. patent application Ser. No. 60/217,227 (Atty Docket No.: CAMIP004P). These patent applications describe models for CYP3A4 that have been built to estimate the stability of organic compounds (potential therapeutics) and predict the likely site of metabolism on the molecule. Such models employ quantum chemical modeling to estimate the intrinsic electronic reactivity of each atom in a molecule. These models have proved to work very well.

Quantum chemical techniques attempt to model the electronic configurations and energies associated with atomic orientations. Approximate geometries can be optimized to stable geometries by minimizing the energy with respect to the atomic coordinates. Reactions can be modeled by transforming the reactant geometry to the product geometry and minimizing all but one degree of freedom.

The essence of a quantum chemical method involves calculating the electronic structure of a given atomic configuration. The electronic configuration of a molecule is obtained by combining atomic orbitals to form molecular orbitals. The equations for the electronic waveforms have been around since the beginning of the twentieth century, but they are not amenable to solution. Therefore different approximations such as semi-empirical methods (using experimental data) and ab initio methods (using a basis set of Gaussian functions to approximate atomic orbitals) are used in the solution to these equations.

Some models disclosed in the above references calculate an intermediate radical structure or otherwise use a quantum chemical analysis to predict metabolic reactivity for every site in order to generate a ranking. Performing such quantum chemical calculations can consume significant computational resources. For some medium and large molecules, these calculations can represent a significant bottleneck in estimating intrinsic electronic reactivity. While this may not represent an inconvenience when only a few compounds are analyzed, it can represent a significant obstacle to analyzing vast libraries of compounds.

Obviously, faster techniques for accurately estimating the reactivity of sites would improve computational throughput. In some cases, the faster techniques would not have to be highly accurate. If the fast techniques could easily identify sites that are unlikely to be metabolized, then those sites could be eliminated from consideration before a more involved quantum chemical calculations are performed on the remaining sites. In this manner, time could be saved by not performing the quantum chemical calculations on the unimportant sites.

In view of the foregoing, the development of accurate but less computationally intensive models to substrate metabolism, and in particular those models that can be used to predict cytochrome p450, would be highly beneficial.

### SUMMARY OF THE INVENTION

The present invention relates generally to methods for developing models used to rapidly predict metabolic stability and regioselectivity of drug molecules. The invention also relates to the models themselves. Training sets, based on a sample of molecules with known reaction rates and/or activation energies, are used along with structural descriptors of the molecules in order to develop mathematical models of metabolism based on regression analysis of the activation energies and descriptors. The resulting models are then used to predict the metabolism of other molecules. The invention is particularly useful in developing simple models of cytochrome p450 enzyme metabolism. Running the mathematical models of this invention is far less computationally intensive than running corresponding quantum-mechanical models.

In a typical approach, the reactivities of one group of substrate molecules can be determined with high accuracy by using relatively slow quantum chemical models. This information is then used to generate a relatively simple model of this invention using appropriate structural descriptors. The reactivities of sites on other molecules, typically ones that are structurally related to the members of the first group, can then be rapidly calculated using the simple models of this invention.

Once a model has been generated in accordance with this invention, it can be used alone, without resort to any quantum chemical analysis, to predict the reactivities of interesting molecules. It can also be used in conjunction with, or to supplement the more rigorous quantum chemical models. One example of the latter approach is to first use a model of this invention to classify all the reactive sites of a molecule. Those sites clearly predicted by the model to be inconsequential are then disregarded. Those sites that appear more interesting can then be more carefully analyzed using a rigorous quantum chemical model. The quantum chemical model can also be used as a sort of check to verify the accuracy of the current invention at a particular reactive site or sites. In a similar manner, it can be used to verify the accuracy of the current invention over a whole class of molecules, for instance, by comparing a QM analysis of some sample molecules within a class with the reactivities calculated by the current invention. In these cases and in others, the savings in time and computational effort can be substantial.

In a preferred embodiment of the invention, a set of fragment or geometry descriptors is applied to a training set of substrate molecules. The activation energies and reactivities of the substrate molecules are predetermined from an external method, such as actual experimental measurements or more computationally intensive estimates, e.g., quantum chemical modeling. The reactive sites of the substrate molecules are described in terms of the descriptors chosen, and a linear regression analysis or other fitting is done to create a simple relationship between descriptor values to reaction rate. In a specific embodiment, the relationship is a linear equation with coefficients that match the reactivity data of the training set with a least squares fit. The linear equation is then applied to subsequent substrate molecules to model and predict their activation energies and reactivities. In another approach, the descriptors are molecular fragments. These are stored with associated values of reactivity. The reactivity values obtained by either approach may be corrected with correction factors such as steric correction factors.

One aspect of the invention provides a method for calculating a fit for a set of site-specific organic chemical descriptors and associated activation energies. The method may be characterized by the operations of identifying reactive sites of the substrate molecules, obtaining activation energy values (or other measures of reactivity) for those molecules, determining the descriptor values that accurately describe each of the reactive sites, and generating an expression for reactivity as function of descriptor values by fitting the reactivity/descriptor data points. Often a simple linear regression technique will yield the desired equation for reactivity. The method provides for organic chemical descriptors such as information about a site atom, neighbor atoms, partial charge, total charge and bond length. The equation generated by the method is to be used to predict the reactivity of substrate molecules having unknown reactivities. The method provides for modeling and predicting the reactivity of substrate molecules with respect to cytochrome p450 metabolism.

Another aspect of the invention provides for a method for predicting the reactivity of a substrate molecule with the operations of identifying reactive sites of the substrate molecules, characterizing the reactive sites based on organic chemical descriptors, using the organic chemical descriptors in a simple equation to model and predict the reactivity of subsequent substrate molecules. In this approach, the organic chemical descriptors are the same descriptors used to derive the linear regression equation from a training set of substrate molecules. The predicted reactivity of a substrate molecule may be adjusted with a steric correction factor.

Another aspect of the invention provides for a computer program product including a machine readable medium for carrying out program instructions pertaining to the above described methods.

These and other features of the present invention will be described in more detail below in the detailed description of the invention and in conjunction with the following figures.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a schematic illustration of the mammalian cyctochrome p450 catalytic cycle, including the non-metabolic decoupling reactions.

FIG. 2 is a schematic illustration of a substrate molecule (drug) with several reactive sites.

FIG. 3 is a process flow diagram depicting some operations that can be performed as part of a process to generate models in accordance with this invention.

FIG. 4A lists, according to a preferred embodiment, sample fragment descriptors for the aliphatic model of oxidation. FIG. 4B lists, according to a preferred embodiment, sample fragment descriptors for the aromatic model of oxidation.

FIG. 5A lists sample coefficients for the aliphatic descriptors of FIG. 4A. FIG. 5B lists sample coefficients for the aromatic descriptors of FIG. 4B.

FIG. 6 presents a hypothetical metabolic profile that graphically represents regioselective site labilities generated in accordance with this invention.

FIG. 7 is high-level flowchart for predicting the metabolic rate of a substrate molecule, starting with the substrate's molecular structure.

FIGS. 8A and 8B illustrate a computer system suitable for implementing embodiments of the present invention.

FIG. 9 is a block diagram of an Internet based system for predicting metabolic properties of molecules in accordance with an embodiment of the present invention.

### DETAILED DESCRIPTION

In the following detailed description of the present invention, numerous specific embodiments are set forth in order to provide a thorough understanding of the invention. However, as will be apparent to those skilled in the art, the present invention may be practiced without resort to these specific details or by using alternate elements or processes. Often well known processes, procedures and components have not been described in detail so as not to unnecessarily obscure aspects of the present invention.

A. INTRODUCTION

The present invention pertains to methods, apparatus, and program code that use simple, rapidly executing models to predict the reactivities of various substrates, and their metabolites and precursors, upon interaction with an enzyme. The methods are most applicable to enzymes with broad substrate specificity (i.e., low substrate selectivity). Examples of such enzymes include monooxygenases (e.g., the CYP enzymes), glucoronyl transferases, and glutathione transferases.

Sample substances for which models of this invention may predict reactivity include various drug compounds or pharmaceutically active agents as well as any molecule introduced (such as by ingestion or inhalation) into a living organism. Upon such introduction, the substances may undergo reactions with various enzymes, including one or more monooxygenase CYP. Substrate reactions with CYP type enzymes include for example hydrogen atom abstraction, aromatic oxidation, and metabolism at heteroatoms. The predominant enzyme mediated reactions discussed herein concern hydrogen atom abstraction and oxygen addition. These reactions specifically include hydroxylation and aromatic oxidation. Obviously, the use of other enzymes such as glucoronyl and glutathione transferases would focus on other chemical reactions.

To assist in understanding the concepts presented herein, the following simple explanations are provided for some terms. The scope of the invention should not necessarily be limited by the following examples.

A “metabolic enzyme” refers to any enzyme that is involved in xenobiotic metabolism. Many metabolic enzymes are involved in the metabolism of exogenous compounds. Metabolic enzymes include enzymes that metabolize drugs, such as the CYP enzymes, uridine-diphosphate glucuronic acid glucuronyl transferases and glutathione transferases.

“Xenobiotic metabolism” refers to any and all metabolism of foreign molecules that occurs in living organisms, including anabolic and catabolic metabolism.

A “reactive site” refers to a site on a substrate molecule that is susceptible to metabolism and/or catalysis by an enzyme. It is to be distinguished from a “active site,” which is the region of an enzyme that is involved in catalysis.

“Reaction rate” refers to the kinetic rate of a chemical reaction or a single step of a chemical reaction. The reaction rate can be predicted by modeling the transition state or estimating the activation energy from the difference in free energy between a substrate and an intermediate form. The term “reaction velocity” is used interchangeably with “reaction rate.”

“Metabolism rate” refers to the overall rate of metabolism of a substrate, regardless of which reactive sites are involved in the metabolism of the drug to a non-reactive form. Thus the reaction rates of all of the reactive sites are involved in determining the metabolic rate.

“Accessibility” refers to the degree to which steric and orientation characteristics of a molecule affect its rate of metabolism and activation energy. “Accessibility correction factors” are factors that quantify these characteristics.

As mentioned, this invention pertains to techniques for generating models that rapidly predict the “reactivity” of a site on an organic molecule. It also pertains the models themselves and their use. As used herein, the term “model” refers to any method or system that can predict reactivity based on chemical structural descriptors. Often a model takes the form of a specific expression for site reactivity and an associated set of descriptors that was chosen for a particular type of reaction (e.g., aromatic oxidation, aliphatic hydrogen atom abstraction, sulfur atom oxidation, etc.).

The models of this invention make use of specific structural descriptors for organic molecules. These descriptors are chosen because they have been discovered to affect site reactivity with high resolution. Particularly interesting descriptors will be described in more detail below. Any organic molecule under consideration, whether used in a training set or an investigation set, is characterized using an appropriate set of descriptors. The descriptor characterization of the molecule is then used to either generate a model (the molecule is part of a training set) or predict reactivity (the molecule is part of an investigation set).

The models of this invention serve as efficient substitutes for full quantum chemical models described in references identified above. As discussed in these references, the quantum chemical models that predict reactivities of sites are particularly useful in predicting the metabolic activity of these sites. Note that most pure quantum chemical models predict “intrinsic” reactivities of sites, without significant regard for steric considerations. This is because, as explained above, most CYP enzymes catalyze oxidation reactions in ways that are only weakly affected by steric effects.

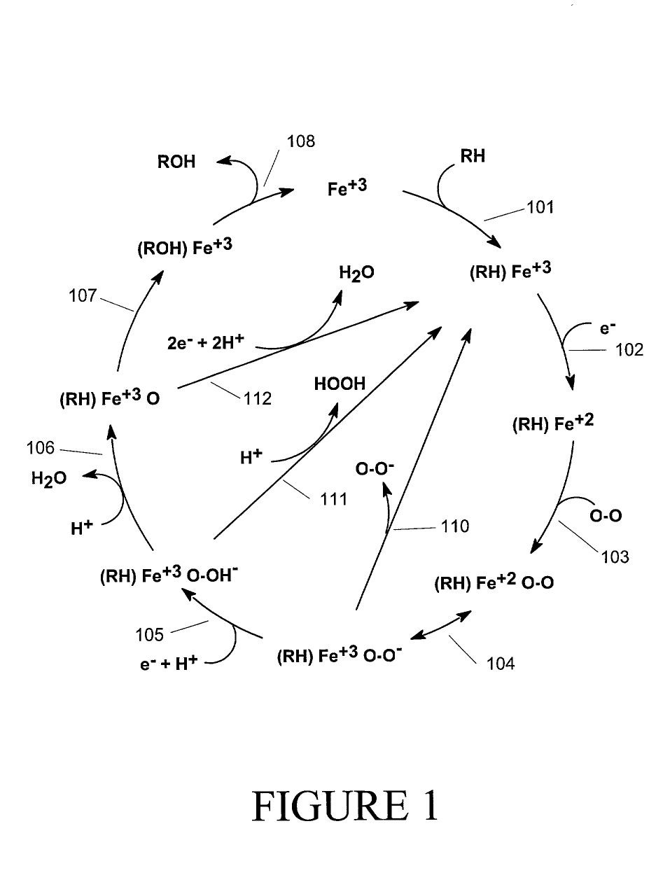

For purposes of context, FIG. 1 illustrates the oxidative hydroxylation catalytic cycle for the mammalian CYP enzyme. The top of the figure shows a generic starting substrate (RH) and generic product (ROH). This hydroxylation reaction is often the first step in metabolizing an exogenous compound, and partly explains the importance of the CYP enzymes in drug deactivation/metabolism.

A first step of the catalytic cycle, 101, shows the initial binding of the substrate to the heme iron atom of the enzyme, which changes the equilibrium spin state of the heme iron from low to high. This lowers the reduction potential of the iron, thus facilitating transfer of an electron from NADPH, via cytochrome p450 reductase, to the iron atom in a second step, 102. In a third step, 103, molecular oxygen binds to the iron atom. In a fourth step, 104, the iron is reduced by one electron and the iron is oxidized from a ferrous state to a ferric state. There is evidence that, at this point, the oxygen can be decoupled from the enzyme as a superoxide ion in a non-metabolic reaction, thus returning enzyme-substrate complex to its initial state in a tenth step, 110. Otherwise, the oxygen is reduced by one more electron in a fifth step, 105, thus forming a peroxy intermediate with the enzyme-substrate complex. Here, a hydrogen peroxide decoupling reaction can take place, an eleventh step, 111, which returns the enzyme-substrate complex to the initial state.

Otherwise, in a sixth step, 106, the peroxide undergoes heterolytic cleavage, with one oxygen leaving the complex as a water molecule and the other oxygen coordinating with the iron atom as a reactive oxygen atom. A water decoupling reaction, a twelfth step, 112, can take the enzyme-substrate complex back to the initial state. Otherwise, the reactive oxygen is transferred to the substrate to form an oxidized product, a seventh step, 107. The product, then dissociates from the enzyme, an eighth step, 108.

Note that the peroxide decoupling reaction, 111 and the water decoupling reaction, 112, both yield the substrate back in its original form in complex with the enzyme. These pathways thus reduce the rate of metabolism of the substrate. If either of the decoupling pathways predominate in the CYP catalytic cycle, then the substrate is unlikely to be metabolized rapidly.

Experimental evidence for the existence of these reaction pathways and intermediates is described in U.S. patent application Ser. No. 09/368,511, by Korzekwa et al. (Atty Docket No.: CAMIP001). That patent application also contains additional material on the mechanisms of CYP enzyme-substrate interaction.

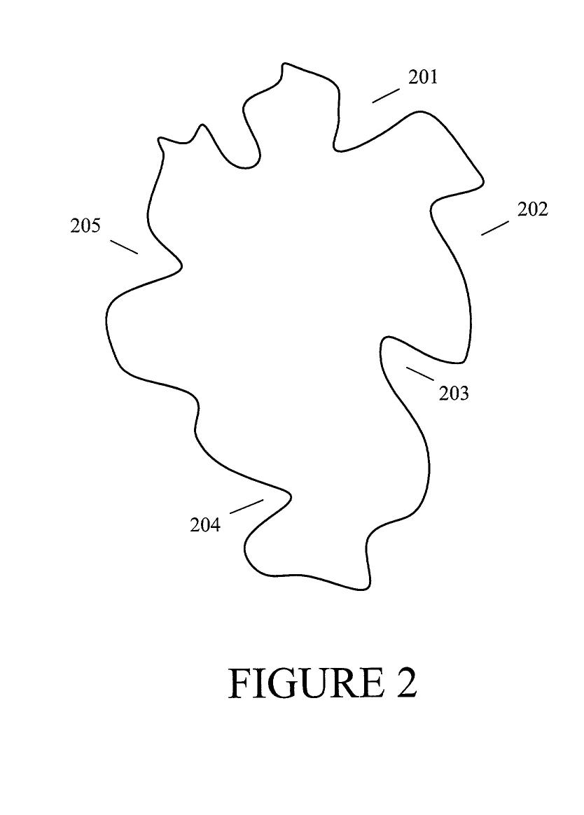

FIG. 2 is a simplified, cartoon illustration of a substrate molecule with several reactive sites, 201-205, for CYP enzyme metabolism. One of the most common ADME/PK problems with a drug candidate is that it is metabolized too quickly. In many cases, an ideal drug would be metabolized slowly enough so that it can be administered about once a day. In the current art, if a drug candidate was being metabolized too quickly for daily administration, the designers of the drug would try to redesign it, typically by modifying the most reactive site in a manner that would make it more stable.

However, changing this most reactive site, even by making it extremely stable or even non-reactive, may or may not result in an appreciable decrease in the rate of metabolism of the drug. The result is essentially unpredictable by methods of the current art. A drug designer much less has the ability to predict how a more minor change in a reactive site will affect the metabolism of the drug. For instance, site 203 might be observed to be the most reactive site. A drug designer could then modify it to make more stable or even unreactive in an attempt to decrease the overall metabolic rate of the substrate. In some instances this will be successful, but if the substrate has one or more reactive sites that also have relatively high reactive rates, then these sites will often “take over” the metabolism of the substrate and the overall metabolic rate will remain essentially unchanged.

Therefore, a drug designer would have to go through the time-consuming process of redesigning one site as essentially a shot in the dark, re-testing the ADME/PK properties, and then redesigning that site and/or one or more of the other reactive sites as additional shots in the dark. After conducting this process on most or all of the reactive sites of the drug, the designer might find that it is essentially impossible to achieve the ADME/PK properties that are desired, particularly without reducing, or perhaps destroying, the desired pharmacological properties of the drug. The chances of altering the pharmacological properties of the drug greatly increase as more and more redesigns of the drug are carried out.

Slowing down the rate of metabolism of a drug candidate is by no means the only ADME/PK property that drug designers try to affect. Alternatively, they may try to speed up the rate of metabolism of drug. In addition, it is generally preferable that a drug have more than one deactivating pathway and/or reactive site, so that chances of dangerous drug interaction, caused by blocking the primary metabolic pathway, are minimized. The CYP enzymes are also susceptible to induction, by which one drug may induce faster metabolism of another drug. The fact that multiple reactive sites are often desirable, for both these reasons, can make the design of the drug even more complicated.

B. GENERATING A MODEL FOR RAPIDLY APPROXIMATING SITE REACTIVITY

1. OVERVIEW

This aspect of the invention may be viewed as a method of producing a model that predicts the lability of reactive sites on a chemical compound. The method may be characterized by the following sequence. First, the implementing system must obtain structural representations for a training set of chemical compounds. Second, for each of these chemical compounds, the system identifies one or more reactive sites pertinent to the model. Then, for each of these reactive sites, the system (i) obtains a lability value from a trustworthy source or technique; and (ii) characterizes the reaction site in terms of values for a plurality of chemical structural descriptors. These descriptors include at least two of the following: an atom type at the reactive site, atoms types at neighboring positions to the reactive site, a partial charge on an atom or group at the reactive site, and a geometric characterization of the reactive site. Finally, for all of said reaction sites, the system uses the lability values and chemical structural descriptor values to obtain an expression for lability that sums contributions from each of the chemical structural descriptors.

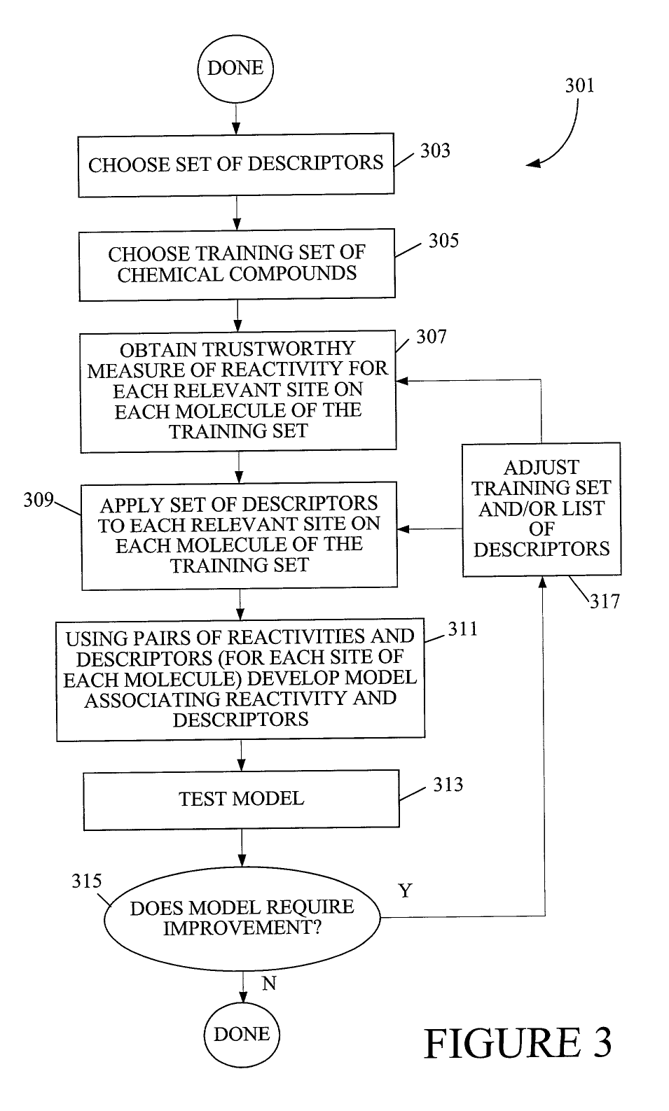

FIG. 3 presents a process flow diagram depicting typical operations that may be employed to generate a model in accordance with an embodiment of this invention. As depicted, a process 301 begins with the choice of an appropriate set of structural descriptors for characterizing organic molecules. See 303. Often, though not always, the set of descriptors is chosen for use in addressing a particular type or class of reactions (e.g., aromatic oxidation). This is because some descriptors are more relevant to one class of reactions, while other descriptors are more relevant to other classes of reactions.

With the model type and associated descriptors chosen, the next process operation involves obtaining information on an appropriate training set of organic molecules. See 305. These molecules are chosen to provide a significant sampling of the types of structural characteristics and reactivities that the model is likely to encounter in practice. For each member of the training set, all potential reactive sites are identified. For example, when the model predicts aromatic oxidation reactions, all aromatic centers of a sample compound are flagged as potential reactive sites. For each of these sites (on each molecule of the training set), the process obtains a trustworthy measure of reactivity. See 307. Typically, this measure of reactivity is calculated using a relatively slow process (at least in comparison to the speed at which the model resulting from process 301 can estimate reactivities). This usually involves modeling the transition state of the reaction using quantum chemical methods. The trustworthy measures of reactivity may be obtained through experimental and/or theoretical techniques.

The measures of site reactivity constitute one component of each data point used to the construct the models of this invention. The other component is the descriptor values. Applying the set of descriptors identified at 303, the process obtains actual values of those descriptors for each site on the training set compounds. See 309. For example, one descriptor may be the partial charge on an atom at the reactive site. The value of the descriptor is the actual numeric value of partial charge at that site. The procedure may obtain these descriptor values by analyzing the simple three-dimensional chemical structures of the members of the training set.

Once the descriptor values have been calculated, each relevant site (e.g., each aromatic center) of each member of the training set is now represented by a set of descriptor values and a trustworthy value of reactivity. Then, using these data points, the process generates the actual model that associates reactivity with the descriptors. See 311. The model may take the form of a simple expression including coefficients for each descriptor value.

With the model in hand, the process may test the model against a particular test set of molecules (or some actual field test molecules). See 313. The molecules used in the test should have known reactivities for their various reactive sites. The ability of the model to accurately predict these reactivities determines whether the model needs improvement. See 315. Assuming that the model does a good job of predicting reactivities, process 301 is complete. Assuming that the model needs improvement, then a revised training set or list of descriptors is chosen. See 317. From there, process control returns to 307 or 309 as appropriate. The revised set or list is chosen to handle the types of molecules or structural features that presented difficulty to the model.

2. CHOOSE A SET OF DESCRIPTORS

These descriptors are chosen to represent ‘important’ structural features of molecules. These features are likely to influence the reactivity (more particularly susceptibility to oxidation) of a particular reactive site on the compound. Generally they may be chosen to capture (a) the classification of the site according to atom type and electronic hybridization, (b) the influence of neighboring atoms and groups, (c) the geometric constraints on the site resulting from participation in a ring, size of the ring, and/or flexibility of the ring, (d) the partial and/or formal charge on the atom at the site (or elsewhere on the molecule).

The influence of neighboring atoms may be captured with descriptors that characterize electron withdrawing properties of neighbors, participation in a conjugation system, participation in a ring system, etc. The geometric state of a site may be captured using descriptors that specify steric factors hindering or facilitating accessibility to a particular site. These factors may result from neighboring structures on the molecule or the relative geometric positioning of a particular site (e.g., at the end of a major axis on an ellipsoid shaped compound). The partial charge on an atom reflects the degree to which the atom donates its electrons to (or receives electrons from) neighboring atoms.

In one specific embodiment, the descriptors identify two or more of following: (a) the particular mechanism of metabolism to occur at the site (e.g., hydroxylation of an aromatic site or hydrogen abstraction from an aliphatic site), (b) the number and types of neighboring atoms, (c) the bond lengths and orders of those atoms, (d) the partial charges on the atoms under consideration, (e) partial charge on an abstracted hydrogen atom, (f) total charge on the site atom(s), (g) hydrogen bond force constants, and (h) derivatives of hydrogen bond force constants.

Detailed examples of descriptor sets used to represent structures relevant to oxidation of certain sites (hydrogen atom abstraction from aliphatic sites, aromatic oxidation, and sulfur atom oxidation) will be presented below. Note that it will often be desirable to employ a set of descriptors that is specific for a particular classes of reactions. For example, one descriptor set may be most optimal for characterizing aromatic oxidation and a different descriptor set may be most optimal for characterizing sulfur oxidation. Examples of fimctional groups, susceptible to oxidation, that may be analyzed using the present invention include C—H, C—C, C≡C, C═C, C═O, C—N, C═N, —S—, —N—, 13 N═, —CHO, —OH, and —C—OH.

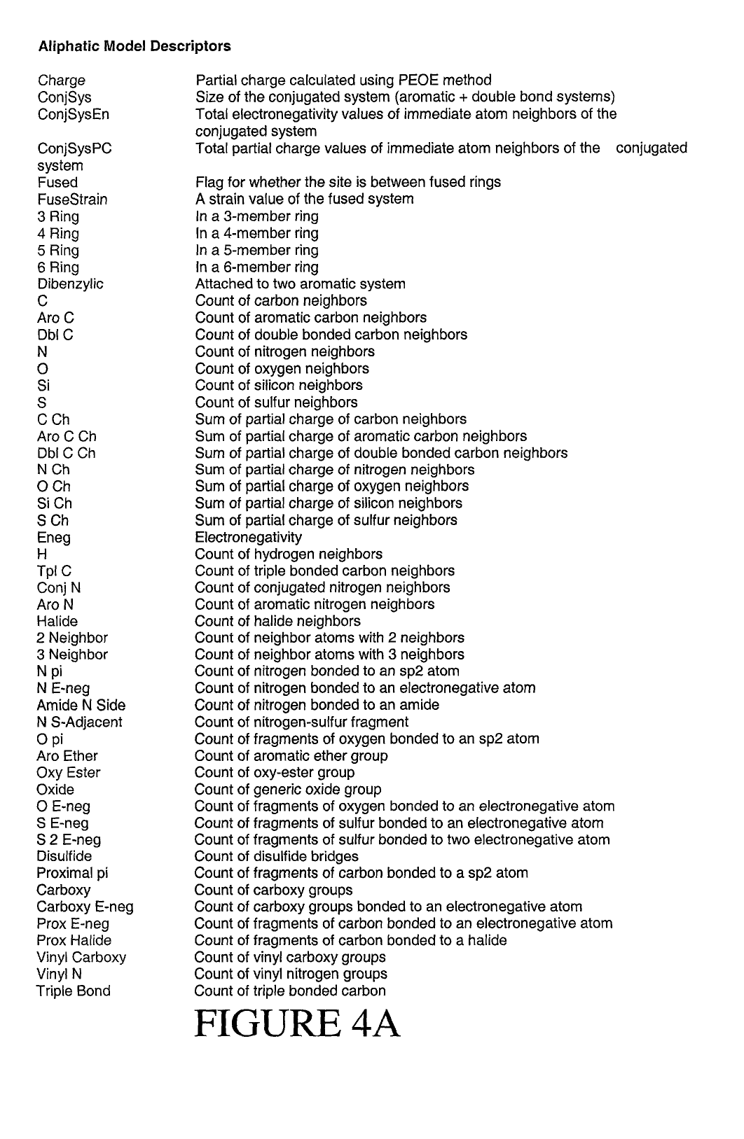

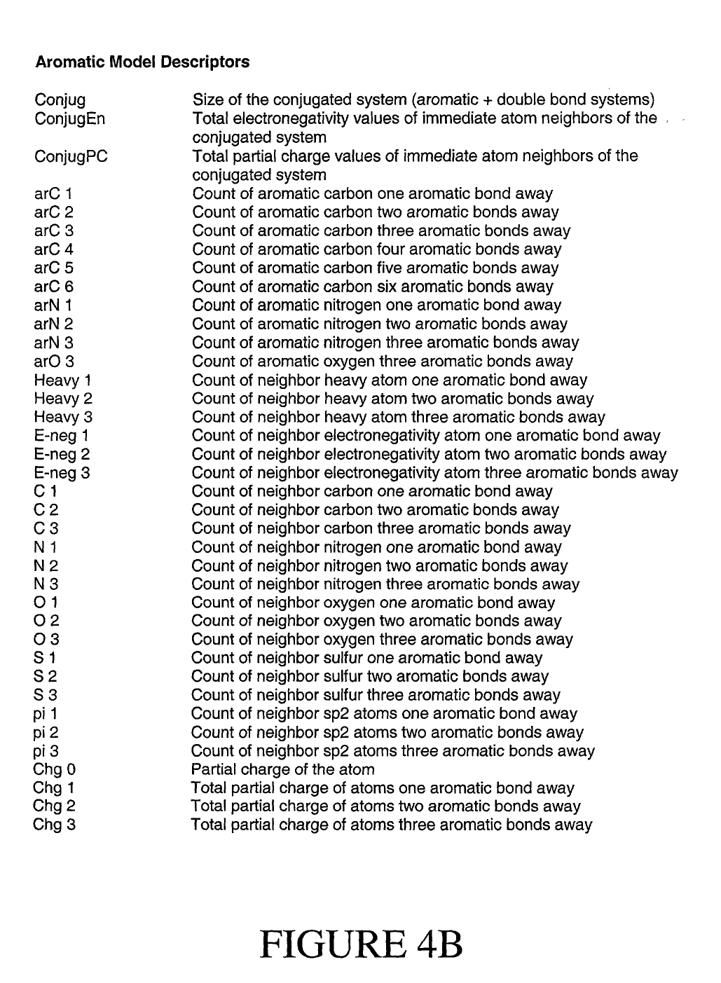

In another specific embodiment, the descriptors identify relevant fragments of a molecule. A system generating such fragments takes, as an input, a molecular structure and applies a set of fragmentation rules. Generally, such rules fragment a molecular structure in a manner that preserves in the resulting fragments the important descriptor information identified above. The reactivities of the fragments obtained from a training set can be stored in a database. If a statistically significant number of reactivities have been computed for a given fragment, and the variance of the values about a mean are within an accepted threshold, then the fragment reactivity is trusted and may be used in place of a quantum chemical calculation of reactivity. FIG. 4A lists example descriptors that can be used with the aliphatic model, with the descriptor in the left-hand column and an explanation of what structural feature the descriptor pertains to in the right-hand column. FIG. 4B does the same for the aromatic model.

3. CHOOSING A TRAINING SET

In developing a model, one should carefully choose a training set. A large group of structurally diverse chemical compounds should be used. Generally, a training set member may be any compound that has been synthesized and has had the reactivities of its sites characterized. The specific compounds chosen for the training set may also be focused on the chemical structural space relevant to the model. Thus, a useful training set may be comprised of compounds that possess an activity related to the activity of the compounds that will ultimately be screened with the model. For example, if the model pertains to drug metabolism, the training set compounds may be known drugs and/or drug-like compounds or other bioactive compounds.

The training set size depends in part on the amount of diversity among the members of the group. Structural “diversity” in the context of this invention means that the compounds of the set have a wide range of different functional groups and functional group environments. Such diversity may be obtained with a wide range of “scaffolds” and “building blocks” and/or a wide range of ring systems, substitutions, etc.

Since this invention pertains to models that predict reactivities of various sites on a compound, the training set should exhibit diversity in the structures of reactive sites represented. As indicated above, the “structure” of a site includes not only the particular atom or moiety at the site, but also the chemical and physical milieu of the site. Thus, for purposes of developing a diverse training set, a diverse set of site structures may include diversity in the neighboring atoms, ring systems, etc.

Often distinct training sets are used for developing separate types of models. Model examples include hydrogen atom abstraction from aliphatic carbon atoms, aromatic oxidation, carbon-carbon double bond oxidation, sulfur oxidation, and nitrogen oxidation. The training set for an aromatic oxidation model should be diverse in the types of aromatic oxidation sites considered. Training set members that have no aromatic character would be irrelevant to such model. Similarly, training sets for nitrogen oxidation models should include various sites for nitrogen atom oxidation. Compounds without nitrogen oxidation sites would not be appropriate in such training sets.

The training set may heavily emphasize groups of compounds and reactive site structures that exhibit widely ranging activation energies—to the extent that such compounds and structures exist. Because the reactivity of such sites may be significantly affected by slight and subtle structural changes, these sites can pose difficulties for the model. Therefore the training set may require numerous similar, but slightly varying, chemical structures.

In one approach to specifying a training set, a group of compounds is selected randomly or systematically based on building blocks, scaffolds, etc. After preliminarily analyzing a group of such compounds, their functional groups may be binned to identify a distribution of functional groups within the original training set. Those compounds that add little if anything to the pool of interesting functional groups may be discarded.

4. CALCULATE ACTIVATION ENERGY FOR EACH RELEVANT SITE

For each site of relevance to the model under development, one must obtain some trustworthy measure of reactivity. Such measure may take various forms but in the end represent the lability or reactivity of a site undergoing an oxidation reaction. Usually this involves modeling the transition state of a site undergoing an oxidation reaction of interest. Of primary interest, the oxidation reaction under consideration is an oxidation reaction catalyzed by CYP enzyme or other catalyst. The site's lability when undergoing such reaction may be calculated using, for example, an activation energy, a change in enthalpy of formation (ΔΔH_{f}) of a reaction intermediate such as a radical, and/or an ionization potential of such radical. These techniques have been described in various references such as U.S. patent application Ser. No. 09/368,511 (Atty Docket No.: CAMIP001), for example.

The transition state energy may be obtained from quantum chemical calculation and/or experimental techniques. Experimental techniques may employ thermodynamic and kinetic data from the reactions of interest to provide and energy of the activated complex (e.g., ΔG^{‡}). For example, the difference in ΔG between two different sites on small molecule substrates of the Cytochrome P450 enzymes can be computed from the difference in measured rates of metabolism of two competing sites. Isotope effect studies of the metabolism at one of the sites can be used to confirm that binding orientation effects are minimal.

Generally, quantum chemical methods for obtaining a measure of reactivity begin with a detailed and accurate three-dimensional electronic representation of the molecule under consideration. Such representation should accurately specify bond lengths and bond angles. They should also specify a detailed specification of electron density. Suitable quantum chemical methods include semiempirical methods and Gaussian-based ab initio methods. These methods are known in the art (see Lickowitz et al., Reviews in Computational Chemistry II, VCH Publishers, 1991, pp. 313-315, which is incorporated herein by reference). To a first approximation, the electronic structure of a molecule is determined by the orientation of atoms in space. Stable geometries of molecules are associated with atomic configurations that provide the lowest energy electronic configurations. Reactions occur through changes in atomic configurations from reactants to products. Along the reaction coordinate, the energy increases from the reactant geometry (stable low energy configuration) to transition state geometry (unstable, high energy configuration) and then decreases again to form products. The challenge typically involves calculating the energy of the transition state.

The quantum chemical approximated activation energy for a hydrogen atom abstraction reaction serves as a suitable measure of reactivity for aliphatic groups undergoing CYP catalyzed oxidation. Similarly, the quantum chemical approximated activation energy for a methoxy addition reaction serves as a suitable measure of reactivity for aromatic groups undergoing CYP catalyzed oxidation.

A C—H site's activation energy for oxidation may be approximated by relating the activation energy to the heat of reaction for a hydrogen atom abstraction reaction and/or the ionization potential of the resulting radical. An aromatic center's activation energy for oxidation may be approximated from the heat of reaction associated with a methoxy addition reaction and/or the ionization potential of the resulting radical.

Various approaches using these methodologies are described in U.S. patent application Ser. No. 09/368,511 (Atty Docket No.: CAMIP001).

As part of the process of ascribing a reactivity to a particular site, one may estimate an enthalpy difference between a compound and a radical produced by removing a hydrogen atom at the site, adding a methoxy group to an aromatic group at the site, or other oxidation mechanism. In one embodiment, enthalpies are calculated using a semi-empirical quantum-chemical modeling program such as AM1 that optimizes a given three dimensional structure to a local energy minimum and calculates electron density distributions from approximate molecular orbitals. The process determines the enthalpy difference between the radical and base form of the molecule. Assuming that the change entropy (ΔS) of the reaction is close to zero, which is a good assumption for the conditions under which CYP oxidation takes place, the process yields a good approximation of the activation energy value (E_{A}) for the reactive site. AM1 is available as part of the public-domain software package MOPAC, which is available from the Quantum Chemistry Program Exchange, Department of Chemistry, Indiana University, Bloomington, Ind. The MOPAC-2000 version of MOPAC can be obtained from Schrödinger, Inc., of Portland, Oreg. As mentioned, the ionization potential of the radical is also used in estimating the E_{A}. It may be calculated using AM1 as well.

5. GENERATING EXPRESSIONS FOR ACTIVATION ENERGY

An expression for site reactivity (e.g., activation energy) may be obtained from any suitable data fitting technique. Generally, the expression is obtained by associating site reactivity with particular structural descriptors. Association represents an attempt to find a relationship between the two groups of variables. One set of variables is the dependent set of variables and these are a function of the other set, the independent set of variables. In this invention, the dependent variables are reactivities or labilities (e.g., trustworthy calculated activation energies) of reactive sites undergoing an oxidation reaction and the independent variables are the structural descriptor values.

Examples of data fitting techniques that may be used with this invention include various regression techniques, partial least squares, principal component analysis, back-propagation neural networks and genetic algorithms. Principal component analysis is described in P. Geladi, Anal Chim. Acta, 1986, 185, 1, which is hereby incorporated by reference.

A linear regression equation relates independent and dependent variables (Y=XB+e where Y is the dependent variable represented by a vector (i.e., reactivity of site of the training set members), X is the independent variable represented by a matrix (i.e., structural descriptors), B is the regression coefficient represented by a vector, and e is the residual). PLS (Projection to Latent Structures or Partial Least Squares) regression analysis is most commonly used with this invention because it can process large numbers of correlating descriptors while minimizing the risk of over-fitting.

In practice, one analyzes each member of the training set. For each member, one considers a list of potential reactive sites. Obviously, the sites of interest include only those that can undergo the reaction of the model at hand. For example, one would not use descriptor values for an aliphatic site to develop a model of aromatic oxidation.

For each relevant site, one must employ (a) a list of descriptors and (b) an activation energy (or other measure of site reactivity such as some combination of ΔΔHf or ionization potential). If the model shows a need for improvement, one might want to “tune” the list of descriptors to improve the model. In any event, with the list of descriptors and measure of reactivity in hand for each site, one applies a regression technique or other fitting routine to obtain an expression for the site reactivity (independent variable) as a function of the descriptors (independent variables). Each site represents a “point” in n dimensional space, where n is one plus the number of descriptors (because the reactivity itself is another dimension).

The form of the expression for reactivity should be chosen to balance accuracy and simplicity. It has been found that first order expressions accurately model activation energies for many sites—when the set of descriptors is chosen as described above. However, there is in principle no reason why other forms of expressions could not be used as well. For example, non-linear functions, higher order polynomial functions, transcendental functions, discontinuous functions, etc. may be applied. Care must be used when deploying such functions, as they may be less stable and more computationally intensive than the simpler first order linear expressions. In any event, the invention is not limited to first order linear expressions for activation energy or other measure of reactivity.

In one embodiment, activation energy of a site can be approximated using expressions of the following form:

Eₐ=Eₐ0+Eₐ1(x1)+Eₐ2(x2)+Eₐ3(x3)+ . . .

In this expression, Eₐ0, Eₐ1 . . . are numerical values of coefficients, Eₐ is the dependent variable (activation energy), and x1, x2 . . . are the independent variables (descriptors).

Preferably, the model employs a single expression for site reactivity to handle all cases within a particular oxidation reaction (model). The concept of a model can be defined broadly or narrowly. In the broadest possibility, a model covers all types of oxidation reactions. In a preferred embodiment, separate models are used for aromatic oxidation, aliphatic hydrogen atom abstraction, carbon-carbon double bond oxidation, sulfur oxidation, and nitrogen oxidation. When multiple models are employed, the algorithm employing the models will have to choose the appropriate model for each reactive site before calculating reactivity. Obviously, more specific models can be used. In one embodiment, multiple equations (models) are generated for a given oxidation reaction. For example, one might have one aromatic oxidation “model” for monocyclic systems, a second aromatic oxidation model for polycyclic systems, yet another aromatic oxidation model for nitrogen or sulfur containing heterocycles, etc.

In some instances, the model may employ “corrections” for orientation, steric hindrance or other “non-intrinsic” effects on site reactivity. Most enzymes other than the CYPs exhibit high specificity for substrates. Even CYPs exhibit some orientation preferences. This specificity can be incorporated into models of this invention. In one embodiment, separate processing logic is developed to bias some of the site specific reactivities initially calculated with the simple descriptor based expressions. One example of such processing logic is presented in U.S. Provisional Patent Application No. 60/217,227 (Atty Docket No.: CAMIP004P), previously incorporated by reference.

C. USING MODELS TO APPROXIMATE SITE REACTIVITY

1. GENERAL METHODOLOGY FOR PREDICTING METABOLIC PROPERTIES

Generally, this aspect of the invention may be viewed as a method for predicting labilities of reactive sites on a chemical compound. Such method may be characterized as follows. First, the implementing system identifies a reactive site on the chemical compound. Second, it identifies values for a plurality of chemical structural descriptors for the reactive site. These descriptors specify at least one of the following: an atom type at the reactive site, atom types at neighboring positions to the reactive site, a partial charge on the atom or group at the reactive site, and a geometric characterization of the reactive site. Third, the system calculates a lability value for the reactive site by summing terms of an expression, wherein the terms include or are derived from the chemical structural descriptors. The first three operations are repeated for more additional reactive sites of the chemical compound. Finally, the system outputs calculated lability values for the reactive sites on the chemical compound. The system may simultaneously display the calculated lability values for all reactive sites on the compound.

Once the models of this invention have been generated, they can execute very rapidly to predict metabolic reactivities of some or all of the sites on a potential therapeutic or other organic molecule. In one embodiment, the models are used alone, without resort to any quantum chemical analysis. In such case, each potential reactive site of a molecule is analyzed by inputting a set of descriptor values for that site and using the model to predict a site-specific reactivity. When all the sites have been analyzed in this manner, the absolute and relative reactivity values are used to draw conclusions about the rate at which the molecule will metabolize.

The models of this invention can also be used in conjunction with, or to supplement, the more rigorous quantum chemical models. One example of the latter approach involves first using the current invention to classify all the reactive sites of a molecule. Those sites that the model clearly identifies as inconsequential are disregarded. Those sites that appear more interesting are more carefully analyzed using a rigorous quantum chemical model. The inconsequential sites may be those that give very low reactivity values.

The quantum chemical model can also be used as a “check” to verify the accuracy of models of the current invention as applied to a particular reactive site or group of sites. In a similar manner, it can be used to verify the accuracy of the current invention over a whole class of molecules, for instance, by comparing a quantum chemical analysis of some sample molecules within a class with the reactivities calculated by the current invention. In these cases and in others, the savings in time and computational effort can be substantial.

According to a specific embodiment, the current invention is used to generate a “metabolic profile” such as the Metabolic Landscape™, of Camitro, Inc., of Menlo Park, Calif. The metabolic profile is generated for each molecule being analyzed. According to a specific embodiment, the metabolic profile contains a calculated lability for each reactive site in a molecule. The reactive sites are typically collected or binned into certain useful categories, such as labile, moderately labile, moderately stabile, and stabile, for example. The reactivities of the reactive sites are typically represented in a visual manner that is useful to the user and makes important information about the molecule readily apparent. For instance, small vertical bars representing each site may be drawn in proportion to the lability of the site, with the important labile sites marked in a visually noticeable color. FIG. 6 presents one example of a metabolic profile that may be generated using the tools of this invention. As shown in this figure, the structure of the compound diltiazem is depicted with various reactive sites highlighted. These sites are characterized in terms of their relative labilities both numerically and in the form of a bar chart.

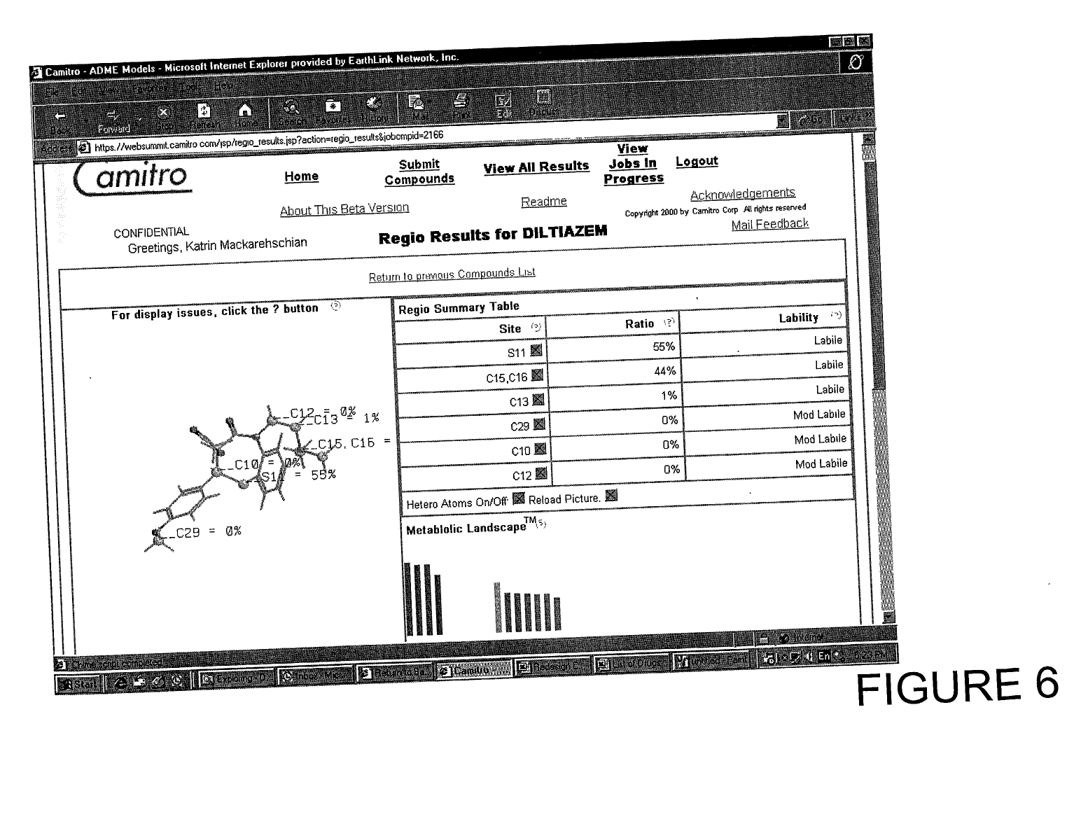

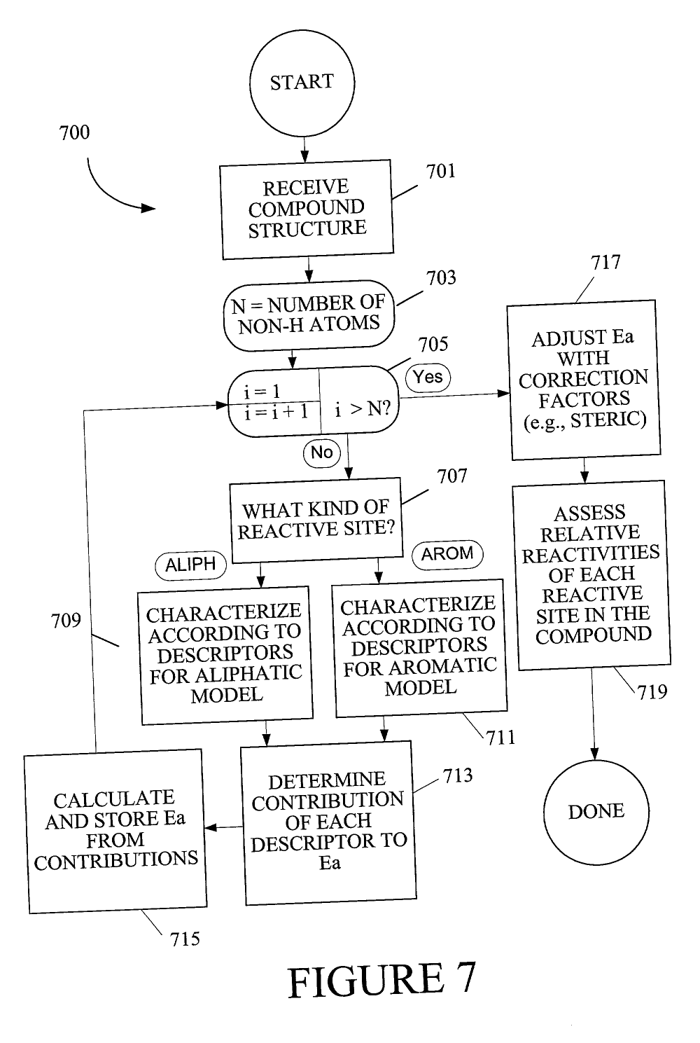

The flowchart of FIG. 7 schematically illustrates from a high-level one preferred process, 700, for predicting the metabolic rate of a substrate molecule. Initially at operation 701, the molecular structure of the substrate is received. The molecular structure can be received as an organic chemistry string of atoms, a two-dimensional structure, a IUPAC standard name, a 3D coordinate map, or as any other commonly used representation. If not already in 3D form, a 3D coordinate map of the molecule is generated, using a geometry program such as Corina or Concord. The 3D structure generator Corina is available from Molecular Simulations, Inc., of San Diego, Calif. and Molecular Networks GmbH of Erlangeh, Germany. Concord is available from Tripos, Inc. of St. Louis, Mo. Corina uses straightforward rules about molecular bond and functional group conformation to generate an approximate geometry 3D structure, which is optimized to a local energy minimum. For instance, if an aniline group is encountered, then it will be placed in a planar conformation, as that group normally exists. Concord applies a similar method, but also uses a limited set of molecular mechanical rules involving branch angles, strain and torsion, to achieve its 3D structure. This approximate 3D geometry structure then, optionally, can be optimized with a more sophisticated modeling tool, typically AM1. As mentioned, AM1 is a semi-empirical quantum-chemical modeling program that optimizes the given 3D structure to that local energy minimum.

In the depicted example, the process then identifies each non-hydrogen atom in the molecule in order to start the analysis, beginning with operations 703 and 705, where the system sets a variable N equal to the number of non-hydrogen atoms to be considered (703) and iterates over those atoms (705). Iterative loop operation 705 initially sets an index value “i” equal to 1. It then determines whether the current value of i is greater than the value of N. If not, it performs various operations to determine the activation energy (E_{A}) for that non-hydrogen atom.

Assuming that another atom remains to be considered, the controlling logic first decides which oxidation model is appropriate to apply to the current atom. See 707. In the embodiment as described in FIG. 7, only two models, aliphatic oxidation (sp³ sites) and aromatic oxidation are described, for purposes of simplicity and illustration. The first model applies to aliphatic carbon atoms. The second model applies to carbon atoms attached to aromatic systems. If this two-model embodiment were actually to be used, then necessarily some atoms of the compound under consideration would be discarded as fitting into neither model. For example, anytime that the process encountered a nitrogen or sulfur atom, the process would ignore that atom and move onto the next atom. In preferred embodiments, however, additional oxidation models such as sulfur oxidation, nitrogen oxidation and carbon-carbon double bond oxidation, are included so that most or all of the non-hydrogen atoms in the substrate molecule can be analyzed.

In operation 709 or 711, according to the model being used (aliphatic or aromatic oxidation respectively), the atom will be described according to the relevant descriptors for that model. The same or similar descriptors that are used to build each model from the relevant training sets are also used as the descriptors here. Depending upon the type of descriptors (e.g. a fragment or a group parameters describing chemical and physical characteristics of a site), different techniques may be employed. After the relevant fragment of descriptor set is generated, corresponding information about the activation energy must be obtained. In the case of a fragment, a look-up operation may be performed to determine the E_{A} of the site defined by the fragment. In the case of a group of parameters, the contribution of each descriptor is considered. See 713. For any given descriptor in this embodiment, there is a corresponding coefficient value. Each coefficient is obtained from a look up table or other source. This is repeated for each descriptor so that an overall E_{A} value for the carbon atom under consideration can be determined. This overall E_{A} value is calculated by combining (e.g. summing) the individual E_{A} descriptor values according to the linear equation or other expression that was derived using the training set. See 715.

For aliphatic carbon oxidation, there are typically one to three possible hydrogen atoms that could be abstracted. Each separate abstraction may have a different activation energy, particularly for atoms on constrained ring systems. Thus, it may be convenient to consider only the oxidation of a single hydrogen atom, from among all hydrogen atoms attached to the carbon atom. For example, it may be desirable to consider only the hydrogen atom having the longest C—H bond. Alternatively, the model can be designed so that it does not distinguish between abstraction reactions for the various hydrogen atoms attached to an aliphatic carbon. For each aliphatic carbon atom, there is a unique set of descriptor parameters (or fragment) and those parameters or fragment provide an associated activation energy. For aromatic oxidation, each unsubstituted aromatic carbon is characterized with appropriate descriptors to generate an approximate activation energy.

Each new atom in the molecule under consideration is considered in its pass through the loop in process 700. The looping continues until all relevant non-hydrogen atoms have been analyzed. After this has been done, the overall E_{A} values for each relevant atom can optionally and preferably be adjusted for steric or enzyme specific effects on oxidation reactions. For example, some sites oxidize more readily than other chemically similar sites simply because of their preferred orientation within the substrate molecule. Many substrates possess overall structural characteristics that bias them toward certain orientations within the enzyme binding site. Further, some sites are more accessible than others. These effects can be accounted for with correction factors, such as non-quantum chemical accessibility factors of the type described in U.S. Provisional Patent Application No. 60/217,227 (Atty Docket No.: CAMIP004P). See 717.

Next, the relative reactivity of each relevant atom, as a function of the EₐS, is assessed and presented. See 719. With the reactivities of all relevant sites in hand, the substrate molecule can be analyzed to see whether it generally has desirable reactive and metabolic properties. The relative reactive rates and absolute reactive rates can then be represented using a metabolic profile as depicted in FIG. 4, for example. Relevant conclusions about the desirability of reengineering the substrate can drawn from such profile. This analysis is described in more detail in U.S. Pat. application 09/613,875 (Atty Docket No.: CAMIP002) and U.S. Provisional Patent Application 60/217,227 (Atty Docket No.: CAMIP004P).

As mentioned, one preferred embodiment employs a “fragment” descriptor set, which characterizes the environment of a reactive site by structural descriptors defining a collection of bonded atoms comprising a fragment of a molecule. Another preferred embodiment employs a “geometry” descriptor set, which describes the environment of the reactive site based on partial charges, bond lengths, and total charge. The partial charges are based on reference Mulliken charges taken from AM1 calculations in similar environments. The total charge is an approximated total charge in the reactive site environment. Either or both of these approaches can be used to supplement a model using a plurality of descriptor parameters describing site atoms and their neighboring atoms.

Both the fragment and geometry embodiments fit into the flowchart description of FIG. 7 above. Many other descriptor sets can be chosen, some being subsets or modifications of the fragment and geometry sets. It has been found that using the fragment-based approach to be described, that an RMS error of about 0.5-0.8 kcal difference between the fragment-based E_{A} and the QM-computed E_{A}. It has been found that using the geometry-based approach to be described, an RMS error of about 1.5 kcal difference between the geometry-based E_{A} and the QM-computed E_{A} has been achieved.

2. CALCULATING SITE REACTIVITY

a. STRUCTURAL DESCRIPTORS USED FOR OXIDATION REACTIONS

For aliphatic carbon oxidation, there are typically one to three possible hydrogen atoms that could be abstracted. Each separate abstraction may have a different activation energy, particularly for sites on constrained ring systems. Thus, it may be ... and those parameters or fragments provide an associated activation energy. For aromatic oxidation, each unsubstituted aromatic carbon is characterized with appropriate descriptors to generate an approximate activation energy.

b. PROCESS OF APPROXIMATING ACTIVATION ENERGY

As indicated above, the actual process of estimating a site's reactivity in accordance with invention involves identifying the appropriate descriptor parameters (fragments or descriptor values of the site under consideration for example) associated with the oxidation reaction of interest. Once these parameters are identified, they are used with the model to obtain a value of reactivity. This may involve looking up the reactivity from a list or database of fragments or descriptor values for example. Alternatively, it may involve identifying a set of coefficients or other parameters used in an expression for reactivity. These coefficients or other equation parameters are matched with the appropriate parameters and then the expression is evaluated to generate the site's reactivity.

In one embodiment, a set of coefficients for the oxidation reaction under consideration is taken from a table. The coefficients are selected for each non-zero descriptor parameter associated with the site under consideration. Note that if there are multiple models for a particular oxidation reaction type, then one would have to first choose the appropriate model in the table (or the table for the model) before pulling out coefficients for the relevant descriptors.

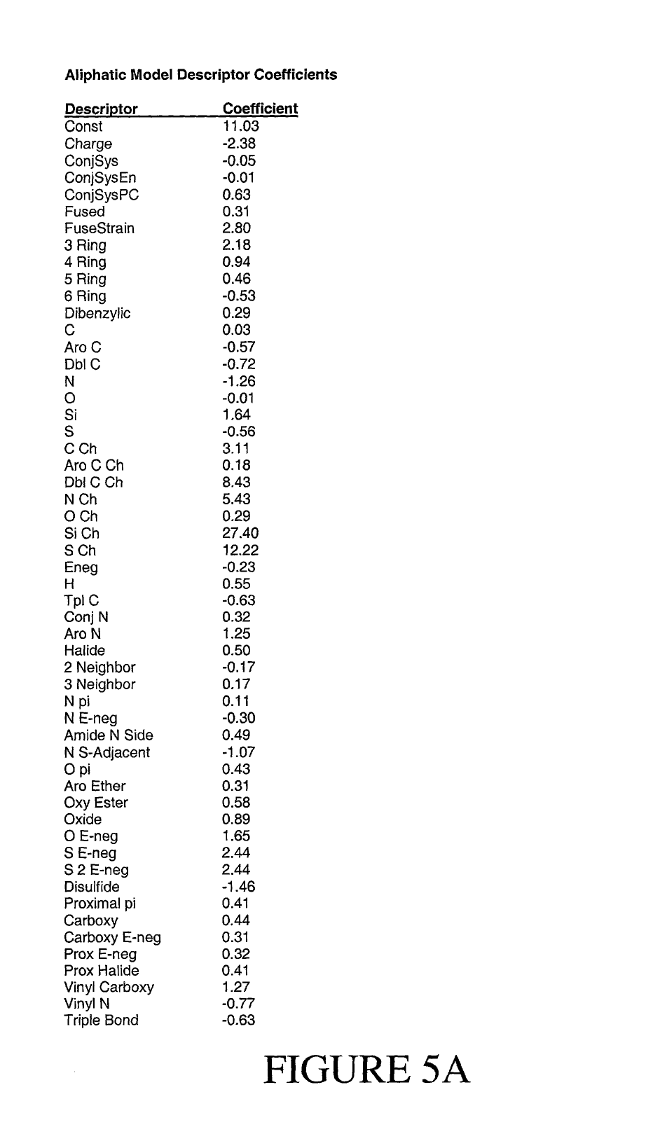

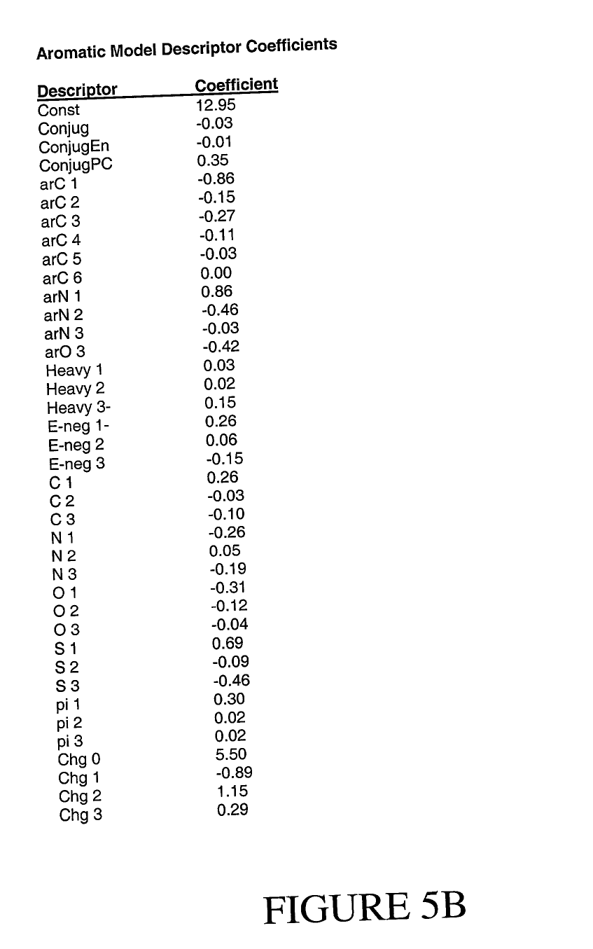

Sample expressions with coefficients are reproduced below for various oxidation reactions. It has been found that when using the fragment-based descriptor approach for aliphatic sites, an RMS error from quantum mechanics based E_{A} values of about 0.8 kcal/mol was achieved (0.5 kcal/mol for aromatic sites). It has been found that when using the geometry-based approach for aliphatic sites, an RMS error from quantum mechanics based E_{A} values of about 1.5 kcal/mol was achieved. FIG. 5A lists, in a preferred embodiment, sample coefficients that correspond to the aliphatic descriptors of FIG. 4A. FIG. 5B does the same for the aromatic descriptors of FIG. 4B.

3. APPLICATIONS AND EXAMPLES

As explained, the models of this invention predict site specific reactivity. This reactivity may represent various types of reaction information. It may represent quantum chemically generated site reactivity, or experimentally generated site reactivity, or some combination of the two. The actual form will typically correspond to the form of the trustworthy reactivity values provided with the training set to generate the model.

The models of this invention may be used for various high throughput applications. For example, the models are useful for processing large chemical libraries derived from combinatorial synthesis. Alternatively, the models can be used for high confidence screens of hits that have been identified by a drug development concern.

For aliphatic sites, the fragment descriptor model can be trained to fit the quantum computed activation energies with a correlation coefficient, r2, of 0.8, and a root-mean-squared error, RMSE, of 0.9 kcal/mol. When applied to data not included in the regression, the r2 is 0.8 and the RMSE is 0.9 kcal/mol, essentially unchanged. For aromatic sites, the fragment descriptor model can be fitted to an r2 of 0.8 and an RMSE of 0.6 kcal/mol. When applied to data not included in the regression, the r2 is 0.5 and the RMSE is 0.5 kcal/mol. When the models are combined with the steric and binding orientation accessibility models described in U.S. Provisional Patent Application 60/217,227 (Atty Docket No.: CAMIP004P), and applied to predicting CYP3A4 metabolism, the results are indistinguishable from using quantum computed activation energies.

D. HARDWARE AND SOFTWARE

Generally, embodiments of the present invention employ various processes involving data stored in or transferred through one or more computer systems. Embodiments of the present invention also relate to an apparatus for performing these operations. This apparatus may be specially constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM devices and holographic devices; magneto-optical media; semiconductor memory devices, and hardware devices that are specially configured to store and perform program instructions, such as read-only memory devices (ROM) and random access memory (RAM), and sometimes application-specific integrated circuits (ASICs), programmable logic devices (PLDs) and signal transmission media for delivering computer-readable instructions, such as local area networks, wide area networks, and the Internet. The data and program instructions of this invention may also be embodied on a carrier wave or other transport medium. Examples of program instructions include both machine code, such as produced by a compiler, and files containing higher level code that may be executed by the computer using an interpreter.

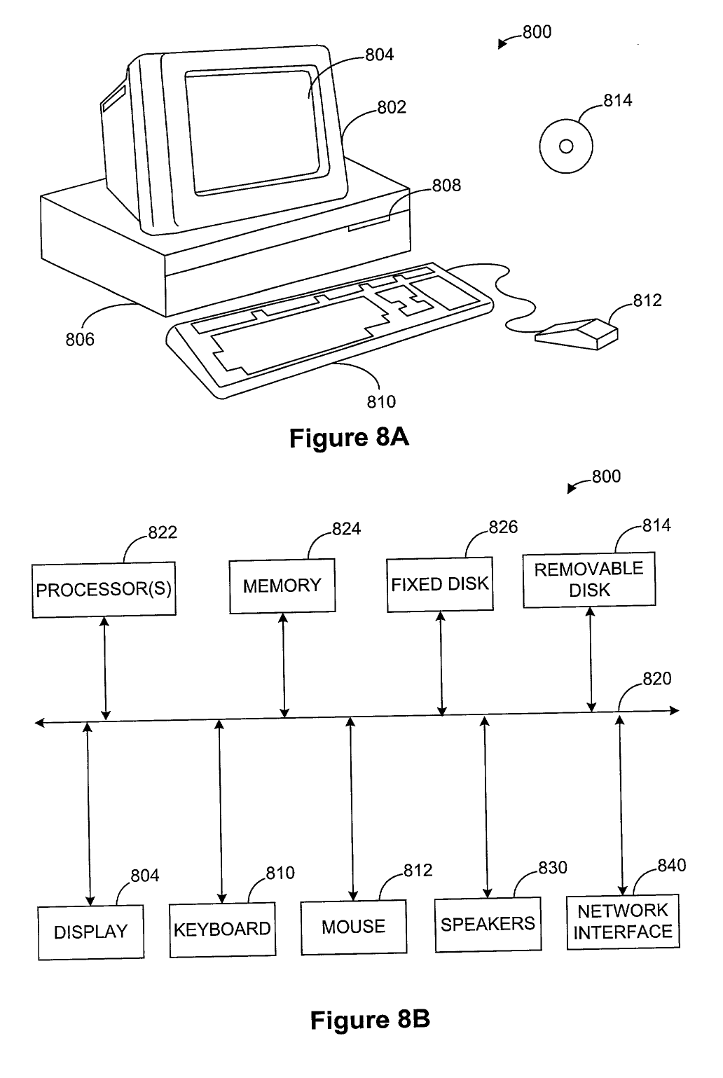

FIGS. 8A and 8B illustrate a computer system 800 suitable for implementing embodiments of the present invention. FIG. 8A shows one possible physical form of the computer system. Of course, the computer system may have many physical forms ranging from an integrated circuit, a printed circuit board and a small handheld device up to a very large super computer. Computer system 800 includes a monitor 802, a display 804, a housing 806, a disk drive 808, a keyboard 810 and a mouse 812. Disk 814 is a computer-readable medium used to transfer data to and from computer system 800.

FIG. 8B is an example of a block diagram for computer system 800. Attached to system bus 820 are a wide variety of subsystems. Processor(s) 822 (also referred to as central processing units, or CPUs) are coupled to storage devices including memory 824. Memory 824 includes random access memory (RAM) and read-only memory (ROM). As is well known in the art, ROM acts to transfer data and instructions uni-directionally to the CPU and RAM is used typically to transfer data and instructions in a bi-directional manner. Both of these types of memories may include any suitable of the computer-readable media described below. A fixed disk 826 is also coupled bi-directionally to CPU 822; it provides additional data storage capacity and may also include any of the computer-readable media described below. Fixed disk 826 may be used to store programs, data and the like and is typically a secondary storage medium (such as a hard disk) that is slower than primary storage. It will be appreciated that the information retained within fixed disk 826, may, in appropriate cases, be incorporated in standard fashion as virtual memory in memory 824. Removable disk 814 may take the form of any of the computer-readable media described below.

CPU 822 is also coupled to a variety of input/output devices such as display 804, keyboard 810, mouse 812 and speakers 830. In general, an input/output device may be any of: video displays, track balls, mice, keyboards, microphones, touch-sensitive displays, transducer card readers, magnetic or paper tape readers, tablets, styluses, voice or handwriting recognizers, biometrics readers, or other computers. CPU 822 optionally may be coupled to another computer or telecommunications network using network interface 840. With such a network interface, it is contemplated that the CPU might receive information from the network, or might output information to the network in the course of performing the above-described method steps. Furthermore, method embodiments of the present invention may execute solely upon CPU 822 or may execute over a network such as the Internet in conjunction with a remote CPU that shares a portion of the processing.

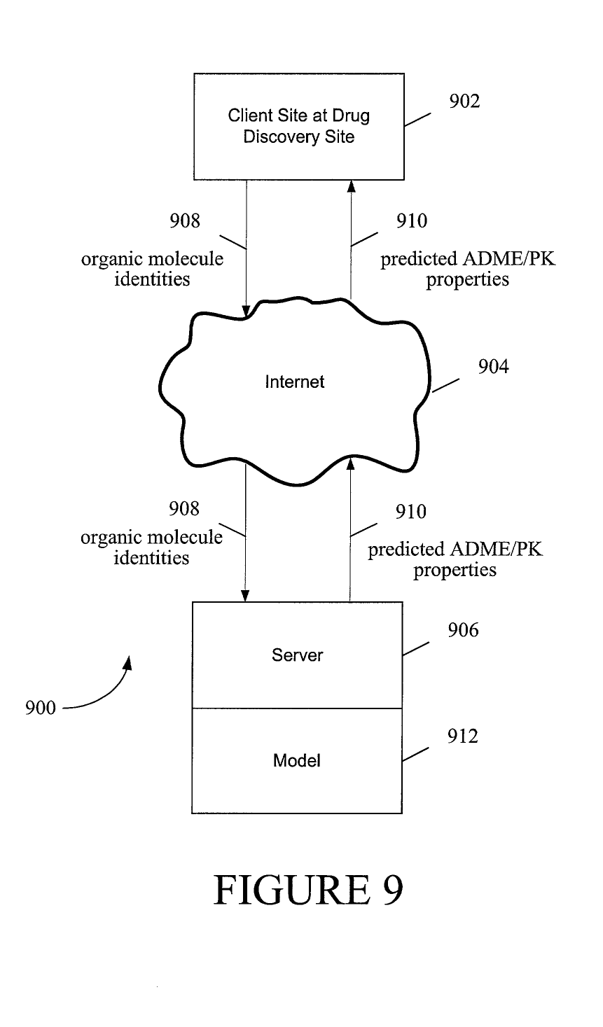

FIG. 9 is a schematic illustration of an Internet-based embodiment of the current invention. See 900. According to a specific embodiment, a client 902, at a drug discovery site, for example, sends data 908 identifying organic molecules 908 to a processing server, 906 via the Internet 904. The organic molecules are simply the molecules that the client wishes to have analyzed by the current invention. At the processing server 906, the molecules of interest are analyzed by a model 912, which predicts site-by-site reactivities in accordance with the current invention. After the analysis, the calculated ADME/PK properties 910, are sent via the Internet 904 back to the client 902. The computer system illustrated in FIGS. 8A and 8B is suitable both for the client 902 and the processing server 906. In a specific embodiment, standard transmission protocols such as TCP/IP (transmission control protocol/internet protocol) are used to communicate between the client 902 and processing server 906. Standard security measures such as SSL (secure socket layer), VPN (virtual private network) and encryption methods (e.g., public key encryption) can also be used.

Although various details have been omitted for brevity's sake, many design alternatives may be implemented. Therefore, the above examples are to be considered as illustrative and not restrictive, and the invention is not to be limited to the details given herein, but may be practiced within the scope of the appended claims.

What is claimed is:

1. A method of producing a model that predicts the lability of reactive sites on a chemical compound, the method comprising:
(a) obtaining structural representations for a training set of chemical compounds;
(b) for each of said chemical compounds, identifying one or more reactive sites pertinent to the model;
(c) for each of said reactive sites,
(i) obtaining a lability value from a trustworthy source or technique; and
(ii) characterizing the reactive site in terms of values for a plurality of chemical structural descriptors, including at least two of an atom type at the reactive site, atoms types at neighboring positions to the reactive site, a partial charge on an atom or group at the reactive site, and a geometric characterization of the reactive site; and
(d) for all of said reactive sites, using the lability values and chemical structural descriptor values to obtain an expression for lability that sums contributions from each of the chemical structural descriptors.

2. The method of claim 1, wherein the structural representations are three dimensional depictions including at least bond lengths and bond angles.

3. The method of claim 1, wherein the model predicts lability for an oxidation reaction selected from the group consisting of aromatic oxidation, aliphatic hydrogen atom abstraction, carbon-carbon double bond oxidation, nitrogen atom oxidation, and sulfur atom oxidation.

4. The method of claim 3, wherein identifying reactive sites pertinent to the model comprises identifying sites where the oxidation reaction can occur on the chemical compounds of the training set.

5. The method of claim 1, wherein obtaining a lability value from a trustworthy technique comprises calculating at least one of an activation energy, an ionization potential of an intermediate radical formed at a reactive site under consideration, and a delta enthalpy of formation for the intermediate radical formed at the reactive site under consideration versus the base chemical compound under consideration.

6. The method of claim 5, wherein the trustworthy technique employs a quantum mechanical representation of the chemical compounds of the training set.

7. The method of claim 1, wherein the model is an aliphatic hydrogen abstraction model and wherein the descriptors comprise fragment-based descriptors or geometry-based descriptors.

8. The method of claim 1, wherein the model is an aromatic oxidation model, and wherein descriptors comprise fragment-based descriptors or geometry-based descriptors.

9. The method of claim 1, wherein using the lability values and the chemical structural descriptor values to obtain an expression for lability comprises employing a regression technique.

10. The method of claim 9, wherein the regression technique is selected from the group consisting of partial least squares, principal component analysis, and linear regression techniques.

11. A method implemented on a computing device for predicting labilities of reactive sites on a chemical compound, the method comprising:
(a) identifying a reactive site on the chemical compound;
(b) identifying values for a plurality of chemical structural descriptors for the reactive site, said chemical structural descriptors specifying at least one of an atom type at the reactive site, atom types at neighboring positions to the reactive site, a partial charge on the atom or group at the reactive site, and a geometric characterization of the reactive site;
(c) calculating a lability value for the reactive site by summing terms of an expression, wherein the terms include or are derived from individual ones of the chemical structural descriptors;
(d) repeating (a)-(c) for more additional reactive sites of the chemical compound; and
(e) outputting lability values calculated at (c) for the reactive sites on the chemical compound.

12. The method of claim 11, wherein the reactive site is susceptible to one of the following oxidation reactions: aliphatic hydrogen atom abstraction, aromatic oxidation, carbon-carbon double bond oxidation, nitrogen oxidation, and sulfur oxidation.

13. The method of claim 12, wherein the chemical structural descriptors are specific for the oxidation reaction to which the reactive site is susceptible.

14. The method of claim 11, wherein the chemical structural descriptors are selected from the group consisting of an atom type of the reactive site, atom types at neighboring positions of the reactive site, a partial charge on an atom or group at the reactive site, a geometric characterization of the reactive site, and combinations thereof.

15. The method of claim 11, wherein the expression is a linear expression for the lability value and having a defined coefficient for each chemical structural descriptor in the expression.

16. The method of claim 12, wherein the expression is specific for the oxidation reaction to which the reactive site is susceptible.

17. The method of claim 11, wherein the lability value represents an activation energy of an oxidation reaction at the reactive site, an ionization potential of an intermediate radical generated by the oxidation reaction at the reactive site, or a delta enthalpy of formation of the intermediate radical formed by the oxidation reaction at the reactive site.

18. The method of claim 11, further comprising simultaneously displaying the lability values calculated at (c) for all reactive sites.

19. The method of claim 11, wherein outputting the lability values calculated at (c) comprises sending the lability values to a remote network site from which a request to predict lability values has originated.

20. The method of claim 11, further comprising correcting the lability values calculated at (c) by modifying said lability values to account for one or more particular reaction characteristics of one or more cytochrome P450 enzymes.

21. A computer program product comprising a machine readable medium on which is provided programmed instructions for producing a model that predicts the lability of reactive sites on a chemical compound, the programmed instructions comprising:
(a) program code for obtaining structural representations for a training set of chemical compounds;
(b) for each of said chemical compounds, program code for identifying one or more reactive sites pertinent to the model;
(c) for each of said reactive sites,
(i) program code for obtaining a lability value from a trustworthy source or technique; and
(ii) program code for characterizing the reactive site in terms of values for a plurality of chemical structural descriptors, including at least two of an atom type at the reactive site, atoms types at neighboring positions to the reactive site, a partial charge on an atom or group at the reactive site, and a geometric characterization of the reactive site; and
(d) for all of said reactive sites, program code for using the lability values and chemical structural descriptor values to obtain an expression for lability that sums contributions from each of the chemical structural descriptors.

22. The computer program product of claim 21, wherein the structural representations are three dimensional depictions including at least bond lengths and bond angles.

23. The computer program product of claim 21, wherein the model predicts lability for an oxidation reaction selected from the group consisting of aromatic, oxidation, aliphatic hydrogen atom abstraction, carbon-carbon double bond oxidation, nitrogen atom oxidation, and sulfur atom oxidation.

24. The computer program product of claim 23, wherein the program code for identifying reactive sites pertinent to the model comprises program code for identifying sites where the oxidation reaction can occur on the chemical compounds of the training set.

25. The computer program product of claim 21, wherein the program code for obtaining a lability value from a trustworthy technique comprises program code for calculating at least one of an activation energy, an ionization potential of an intermediate radical formed at a reactive site under consideration, and a delta enthalpy of formation for the intermediate radical formed at the reactive site under consideration versus the base chemical compound under consideration.

26. The computer program product of claim 25, wherein the trustworthy technique employs a quantum mechanical representation of the chemical compounds of the training set.

27. The computer program product of claim 21, wherein the program code for using the lability values and the chemical structural descriptor values to obtain an expression for lability comprises program code employing a regression technique.

28. The computer program product of claim 7, wherein the regression technique is selected from the group consisting of partial least squares, principal component analysis, and linear regression techniques.

29. A computer program product comprising a machine readable medium on which is provided programmed instructions for predicting labilities of reactive sites on a chemical compound, the programmed instructions comprising:
(a) program code for identifying a reactive site on the chemical compound;
(b) program code for identifying values for a plurality of chemical structural descriptors for the reactive site, said chemical structural descriptors specifying at least one of an atom type at the reactive site, atom types at neighboring positions to the reactive site, a partial charge on the atom or group at the reactive site, and a geometric characterization of the reactive site;
(c) program code for calculating a lability value for the reactive site by summing terms of an expression, wherein the terms include or are derived from individual ones of the chemical structural descriptors;
(d) program code for repeating (a)-(c) for more additional reactive sites of the chemical compound; and
(e) program code for outputting lability values calculated at (c) for the reactive sites on the chemical compound.

30. The computer program product of claim 29, wherein the reactive site is susceptible to one of the following oxidation reactions: aliphatic hydrogen atom abstraction, aromatic oxidation, carbon-carbon double bond oxidation, nitrogen oxidation, and sulfur oxidation.

31. The computer program product of claim 30, wherein the chemical structural descriptors are specific for the oxidation reaction to which the reactive site is susceptible.

32. The computer program product of claim 29, wherein the chemical structural descriptors are selected from the group consisting of an atom type of the reactive site, atom types at neighboring positions of the reactive site, a partial charge on an atom or group at the reactive site, a geometric characterization of the reactive site, and combinations thereof.

33. The computer program product of claim 29, wherein the expression is a linear expression for the lability value and having a defined coefficient for each chemical structural descriptor in the expression.

34. The computer program product of claim 30, wherein the expression is specific for the oxidation reaction to which the reactive site is susceptible.

35. The computer program product of claim 29, wherein the lability value represents an activation energy of an oxidation reaction at the reactive site, an ionization potential of an intermediate radical generated by the oxidation reaction at the reactive site, or a delta enthalpy of formation of the intermediate radical formed by the oxidation reaction at the reactive site.

36. The computer program product of claim 29, further comprising program code for simultaneously displaying the lability values calculated at (c) for all reactive sites.

37. The computer program product of claim 29, wherein the program code for outputting the lability values calculated at (c) comprises program code for sending the lability values to a remote network site from which a request to predict lability values has originated.

38. The computer program product of claim 29, further comprising program code for correcting the lability values calculated at (c) by modifying said lability values to account for one or more particular reaction characteristics of one or more cytochrome P450 enzymes.

39. A method for calculating a linear regression equation with a set of organic chemical descriptors, each descriptor having a coefficient, the method comprising:
(a) identifying the reactive sites on each substrate molecule in a training set of substrate molecules;
(b) obtaining activation energy and reactivity values for each of the reactive sites from an external method;
(b) determining the organic chemical descriptors that describe each of the reactive sites; and
(c) calculating the coefficients and the linear regression equation.

40. The method of claim 39 wherein the organic chemical descriptors comprise site atom descriptors and neighbor atom descriptors.

41. The method of claim 39 wherein the organic chemical descriptors comprise partial charges, total charge and bond length descriptors.

42. The method of claim 39 wherein the linear regression equation is used to model and predict the reactivity of other substrate molecules.

43. The method of claim 42 wherein the linear regression equation is used to model and predict the reactivity of other substrate molecules in cytochrome p450 metabolism.

44. The method of claim 42 wherein the substrate molecules are drug candidates.

45. A method for predicting the reactivity of a substrate molecule, the method comprising:
(a) identifying reactive sites on the substrate molecule;
(b) characterizing the reactive sites based on organic chemical descriptors;
(c) using the organic chemical descriptors in a linear regression equation designed to model and predict the reactivity of substrate molecules, in order to predict the reactivity of the substrate molecule;
wherein the organic chemical descriptors are the same descriptors used to derive the linear regression equation from a training set of substrate molecules.

46. The method of claim 45 wherein the organic chemical descriptors comprise site atom descriptors and neighbor atom descriptors.

47. The method of claim 45 wherein the organic chemical descriptors comprise partial charges, total charge and bond length descriptors.

48. The method of claim 45 wherein the reactivity of the substrate molecule is calculated specifically with respect to the cytochrome p450 metabolic cycle.

49. The method of claim 45 wherein the substrate molecules are drug candidates.

50. The method of claim 45 wherein the predicted reactivity of a substrate molecule is adjusted with a steric correction factor.

51. A computer program product comprising a machine readable medium on which is stored program code for calculating a linear regression equation with a set of organic chemical descriptors, each descriptor having a coefficient, the program code specifying instructions for:
(a) identifying the reactive sites on each substrate molecule in a training set of substrate molecules;
(b) obtaining activation energy and reactivity values for each of the reactive sites from an external method;
(b) determining the organic chemical descriptors that describe each of the reactive sites; and
(c) calculating the coefficients and the linear regression equation.

52. A computer program product comprising a machine readable medium on which is stored program code for creating a training set of substrate molecules, each substrate molecule comprising at least one reactive site, and each reactive site comprising a plurality of organic chemical descriptors.

53. A computer program product comprising a machine readable medium on which is stored program code for predicting the reactivity of a substrate molecule, each descriptor term having a coefficient, the program code specifying instructions for:
(a) identifying reactive sites on the substrate molecule;
(b) characterizing the reactive sites based on organic chemical descriptors;
(c) using the organic chemical descriptors in a linear regression equation designed to model and predict the reactivity of substrate molecules, in order to predict the reactivity of the substrate molecule;
wherein the organic chemical descriptors are the same descriptors used to derive the linear regression equation from a training set of substrate molecules.
